(19) United States
(12) Patent Application Publication
(10) Pub. No.: US 2006/0183725 A1
(43) Pub. Date: Aug. 17, 2006

(54) Pharmaceutical preparation for oral contraception

(76) Inventors: Thomas Graeser, Windischholzhausen (DE); Claus Claussen, Jena (DE)

Correspondence Address: STRIKER, STRIKER & STENBY, 103 EAST NECK ROAD, HUNTINGTON, NY, 11743, US

(21) Appl. No.: 11/351,090

(22) Filed: Feb. 9, 2006

Related U.S. Application Data
(60) Provisional application No. 60/653,181, filed on Feb. 15, 2005.

(51) Int. Cl. A61K 31/56

(52) U.S. Cl. 514/170

(57) ABSTRACT

The pharmaceutical preparation for oral contraception has 28 daily dosage units, of which at least 21 daily dosage units each contain from 1.5 mg to 2 mg of dienogest and from 0.015 mg to 0.02 mg of ethinyl estradiol together in a pharmaceutically acceptable carrier. Seven or fewer daily dosage units contain no effective ingredient. Each daily dosage unit can be a film tablet for oral administration, which has a tablet core and film coating on the tablet core. At least 30% of the dienogest is released from the tablet core preferably in a delayed manner after more than 30 minutes, while at least 70% of the dienogest and ethinyl estradiol are released from the film coating preferably in 30 minutes, as determined by a standard dissolution test.

### CROSS-REFERENCE

U.S. Provisional Application No. 60/653,181, filed Feb. 15, 2005, also discloses the invention described and claimed herein and provides the basis for a claim of priority of invention under 35 U.S.C. 119 (e).

### BACKGROUND OF THE INVENTION

1. The Field of the Invention

The present invention relates to pharmaceutical preparations, which contain 17α-cyanomethyl-17β-hydroxyestra-4,9-dien-3-one (dienogest) and 17α-ethinyl estradiol (ethinyl estradiol) and also relates to the use of this combination for contraception.

2. Related Art

Oral contraceptives comprising a gestagen ingredient and an estrogen ingredient were first marketed more than 60 years ago. Three essential properties characterize the “contraceptive pill”: contraceptive reliability, very good cycle control and a minimum of side effects. The contraceptive reliability is based first on the gestagen ingredient. It causes an inhibition of the gonadotropin release in the pituitary gland and inhibition of ovulation. Furthermore the peripheral action of the gestagen on the endometrium reduces the probability of implantation of the impregnated egg cell and secretion of a viscous secretion in the cervix, which reduces the sperm count in the womb. Also peripheral action on the fallopian tubes should be noted.

The estrogen ingredient amplifies or increases the inhibitory action of the gestagen on ovulation and is responsible for acceptable cycle control. Since the introduction of hormonal contraceptives research has been directed to the development of preparations, which reduce undesirable side effects, such as arterial and venous thromboses and influences on carbohydrate and lipo-metabolism, while maintaining good contraceptive reliability and cycle control. These types of side effects were known from the first generation of oral contraceptives, which contained a higher content of gestagen and estrogen than is necessary for contraception.

WO 98/04269 discloses, among other things, oral administration of a combination of 250 μg to 4 mg of dienogest and 10 μg to 20 μg of ethinyl estradiol for contraception. In order to achieve a substantial reduction of the total amount of steroids administered per cycle, while maintaining good cycle control, a low dosage gestagen/estrogen combination is administered for 23 to 25 days of the 28-day menstruation cycle. However no results are disclosed in this patent, which show that the inventive concept is successful.

WO 01/015701 claims a pharmaceutical composition for oral administration during 21 days of a 28 day menstruation cycle, which contains drospirenone and ethinyl estradiol, also in a low dosage, in which the drospirenone is present in micronized form. A rapid release of the steroids is especially notable.

Steroidal effective ingredients are metabolized by the Cytochrome P450 enzyme system, which is also responsible for degradation of a series of drugs, as is known from the professional and patent literature. Certain drugs or medications, such as barbiturates, antiepileptic drugs and selected virostats, induce these enzyme systems and reduce the effective ingredient concentration of steroids in blood. In order to compensate for this effect the gestagen content must be increased or at least maintained and not reduced, which results in the previously indicated side effects.

### SUMMARY OF THE INVENTION

It is an object of the present invention to provide an improved pharmaceutical composition based on a combination of dienogest and ethinyl estradiol, in which the total dosage of steroids is reduced, while maintaining the balance between dienogest and ethinyl estradiol, which provides effective contraceptive action and good cycle control and counteracts side effects, like further lowering of the effective ingredient level in blood when other medications are taken.

This object and others which will be made more apparent hereinafter are attained by a pharmaceutical preparation containing from 1.5 mg to 2.0 mg of dienogest and 0.015 mg to 0.020 mg of ethinyl estradiol, which is administered for at least 21 days of a 28-day menstruation cycle.

The effective ingredient combination of dienogest and ethinyl estradiol is generally introduced into pharmaceutical preparations with one or more pharmaceutically acceptable carriers or medicinal carriers. Embodiments of the pharmaceutical preparation according to the invention, which are suitable for oral administration, are in the form of tablets for oral administration, which contain 2 mg of dienogest and 0.020 mg of ethinyl estradiol, 2 mg of dienogest and 0.15 mg of ethinyl estradiol or 1.5 mg dienogest and 0.15 mg of ethinyl estradiol.

The dienogest has a rapid in-vitro release stage and a delayed in-vitro release stage and the ethinyl estradiol has a conventional rapid in-vitro release stage in the pharmaceutical preparations according to the invention. The delayed release fraction of the dienogest amounts to at least 5%, preferably at least 30% of the dienogest, which is released delayed more than 30 min, and more preferably at least 50% delayed more than 30 min, as measured with the dissolution test according to Ph Eur performed by means of a rotating bottle apparatus using 1000 ml of water at 37° C. as dissolution medium and with stirring at stirring speed of 50 rpm. The rapid in-vitro release of the dosage of ethinyl estradiol and dienogest from the film coating occurs to at least 75% in at most 45 minutes, preferably to 70% in 30 min, as determined with the previously indicated dissolution test.

A film tablet with an effective ingredient-containing film coating is one embodiment of the pharmaceutical preparation. The film tablet comprises a tablet core containing dienogest of the second stage and a film coating containing dienogest of the first stage and the entire amount of ethinyl estradiol.

It was surprisingly found that that the partially delayed release of the dienogest from the tablet preparation made the low dosage of the effective ingredient combination of dienogest and ethinyl estradiol in the inventive amount range possible.

It has been shown that the effective ingredient combination in the pharmaceutical preparation according to the invention has anti-androgenic properties besides contraceptive action and thus can be used for prophylaxis and therapy of androgen-induced conditions, such as acne.

In suitable embodiments of the pharmaceutical preparation according to the invention the number of the daily dosage units containing the effective ingredients amounts to 22, 23, 24 or 25 daily dosage units and the number of effective-ingredient free daily dosage units amounts then to 6, 5, 4 or 3 in the 28-day menstrual cycle.

The combination of 1.5 mg to 2.0 mg of dienogest and 0.015 mg to 0.020 mg of ethinyl estradiol can be used for making a pharmaceutical preparation or for preparation of a pharmaceutical composition according to the invention for inhibition of ovulation.

### BRIEF DESCRIPTION OF THE DRAWING

The objects, features and advantages of the invention will now be illustrated in more detail with the aid of the following description of the preferred embodiments, with reference to the accompanying figures in which:

FIG. 1 is a graphical illustration of the respective release profiles for dienogest (labeled DNG) and ethinyl estradiol (labeled EE) from a film tablet preparation according to the invention as described above according to the dissolution test; and

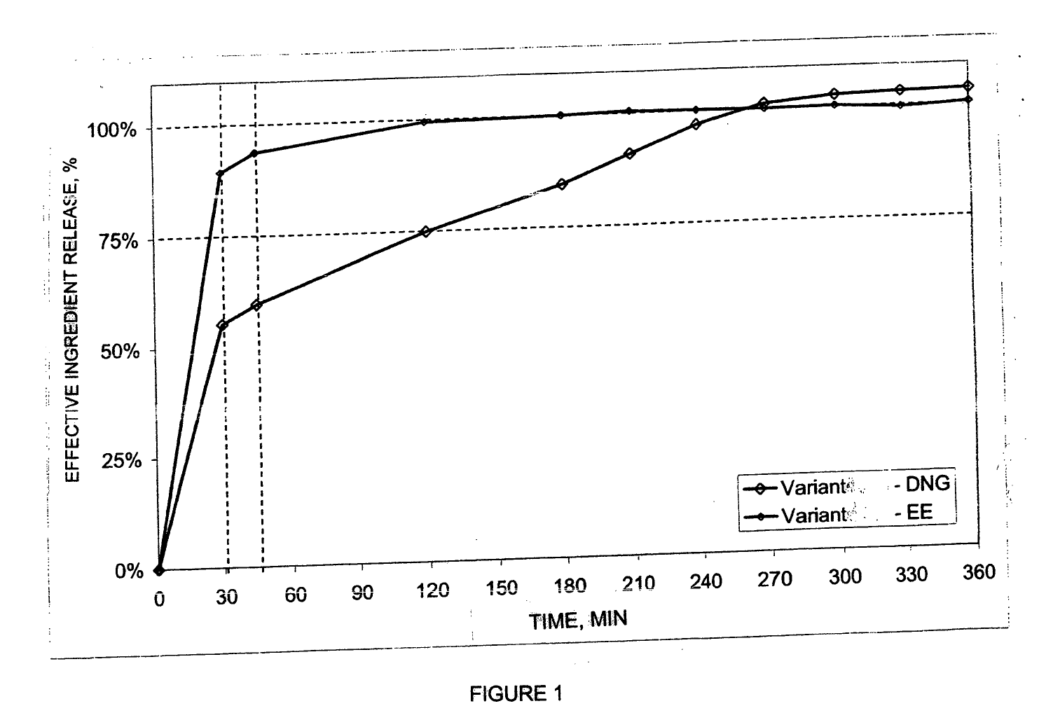

FIG. 2 is a graphical illustration of several release profiles for dienogest (DNG) and ethinyl estradiol (EE) from a number of embodiments of the film tablets according to the invention, as described above according to the dissolution test.

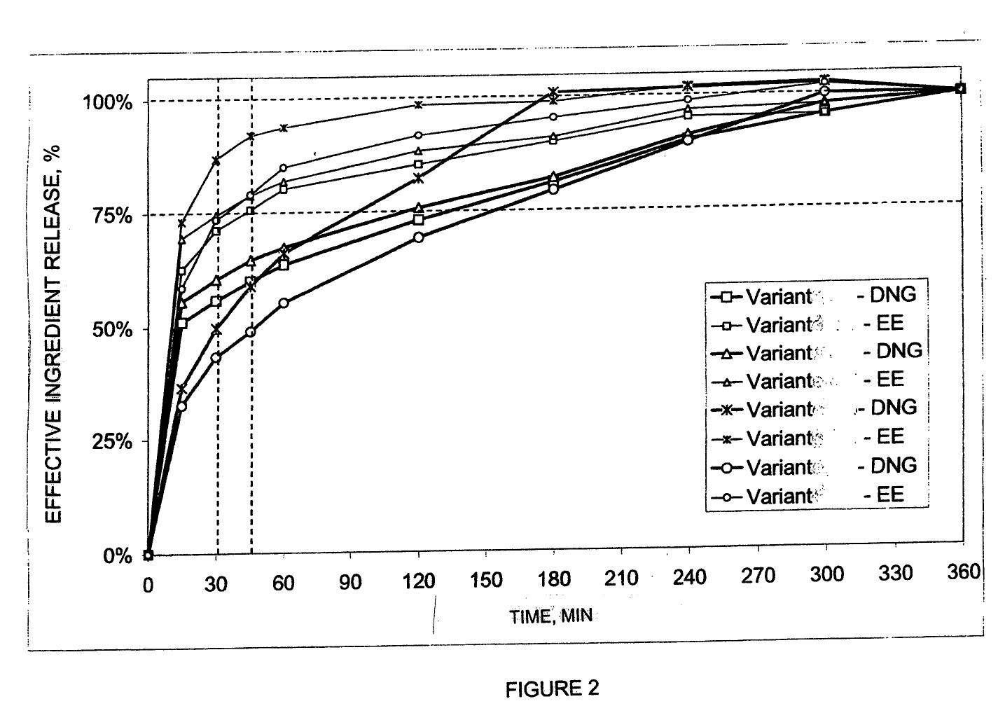

### EXAMPLES

### Clinical Tests of Contraceptive Action of Preparations Containing Dienogest And Ethinyl Estradiol

In a randomized, open clinical study forty women between the ages of 18 to 35 years, who gave written consent to their participation in the study or tests, were treated with two different preparations, which contained dienogest and ethinyl estradiol. The first preparation (A) corresponded to a combination of 2 mg of dienogest (no delayed release) and 0.02 mg ethinyl estradiol. The second preparation (B) comprised a combination of 1.5 mg of dienogest (at 50%=0.75 mg delayed release) and 0.02 mg ethinyl estradiol.

The clinical tests included a pre-treatment cycle (wash-out stage), three treatment cycles and an after-treatment cycle (follow-up stage).

At fixed time points (before start of the first treatment cycle, at the end of the third treatment cycle) different laboratory chemical and diagnostic tests were performed. FSH, LH, estradiol, progesterone, “spinability” and fern phenomenon were determined. The follicle maturation was determined with the help of ultrasound testing procedures. Also SHBG, CBG, total testosterone, triglycerides, cholesterol, HDL, LDL, serum glucose were determined and blood pressure, heart rate, body weight and blood properties were recorded.

The results of the study showed that both preparations reliably inhibited ovulation, which is indicated by the drop of LH, FSH, progesterone and estradiol during the treatment cycle. In both groups follicle growth could be detected by the conducted ultrasound tests, which led to follicle rupture and thus to ovulation (egg release) in no study participants. In three women, who were treated with the preparation A so-called LUFs (luteinized unruptured follicles) were determined, which suggested occurring follicle maturation, but without ovulation. SHBG and CG increased in comparison in both treatment groups. The main parameters influenced by the gestagen content of the preparation were “spinability” and fern phenomenon. The group treated with preparation B tended to have a greater reduction of these parameters than the comparison group. However the difference was not statistically significant. The increase of HDL cholesterol and the drop of LDL cholesterol were significantly greater in treatment group B than in treatment group A. The changes in triglycerides and total cholesterol in both groups were comparable. The glucose tolerance remains largely unaffected in both groups. Total testosterone decreased significantly in both treatment groups and in a comparable manner. The subjective and objective tolerability (lack of side effects) of preparation B was evaluated as good or satisfactory. Also the intensity of regular gestagen withdrawal or deprivation bleeding (in the pill pause phase) was reduced. Furthermore women, who were treated with preparation B, reported fewer premenstrual complaints (headaches, abdominal pains). No differences were found between the groups during the study in regard to blood pressure, heart rate and body weight.

The studies have shown that both preparations have reliably prevented ovulation in all volunteers. It was established that treatment with preparation B increased the tendency for peripheral anti-contraceptive effects. A single undesirable side effect (uncontrolled bleeding, premenstrual complaint) occurred, significantly less often under treatment with preparation B. An improved lipid profile was determined for preparation B.

The results show that, because of a delayed release of dienogest, a low dosage of both dienogest and also ethinyl estradiol (in preparation B) is possible, i.e. the total amount of steroids is reduced, and is appropriate for the requirement of a balance between dienogest and ethinyl estradiol. Similarly good contraceptive action and cycle control are accomplished and side effects, like for example additional lowering of the effective ingredient level in blood when other medications are taken, are compensated. Also preparation A, which contains low dosages of ethinyl estradiol in comparison to conventional preparations, exhibits detectable ovulation inhibiting effects, however not as comprehensive as preparation B which has a low dosage of both dienogest and ethinyl estradiol.

Unless otherwise indicated, all percentages are percentages by weight.

While the invention has been illustrated and described as embodied in a pharmaceutical preparation for oral contraception, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A pharmaceutical preparation for oral contraception, comprising at least 21 daily dosage units for a 28-day menstrual cycle,
wherein each of said daily dosage units comprises 1.5 mg of dienogest, 0.015 or 0.020 mg ethinyl estradiol and at least one pharmaceutically acceptable carrier; or
wherein each of said daily dosage units comprises 2.0 mg of said dienogest, 0.015 mg of said ethinyl estradiol and said at least one pharmaceutically acceptable carrier.

2. The pharmaceutical preparation as defined in claim 1, wherein each of said daily dosage units is a film tablet, said film tablet comprises a tablet core and an effective-ingredent-containing film coating, said film coating contains all of said ethinyl estradiol and a first portion of said dienogest and said tablet core contains a second portion of said dienogest.

3. The pharmaceutical preparation as defined in claim 2, wherein at least 30% of said dienogest is released in a delayed manner from said tablet core after more than 30 minutes and at least 75% of said dienogest and said ethinyl estradiol are released from the film coating in at most 45 minutes, as determined by a dissolution test using water at 37° C. as dissolution medium and with stirring at a 50 rpm stirring speed.

4. The pharmaceutical preparation as defined in claim 2, wherein at least 50% of said dienogest is released from said tablet core in a delayed manner after more than 30 minutes and at least 70% of said dienogest and said ethinyl estradiol are released from the film coating in 30 minutes, as determined by a dissolution test using water at 37° C. as dissolution medium and with stirring at a 50 rpm stirring speed.

5. The pharmaceutical preparation as defined in claim 1, consisting of 22 of said daily dosage units and 6 other daily dosage units, and wherein each of said 22 daily dosage units contains said gestagen and said ethinyl estradiol, each of said other daily dosage units contains no effective ingredients and a total number of said daily dosage units is 28.
